(19) United States
(12) Patent Application Publication
(10) Pub. No.: US 2014/0309291 A1
(43) Pub. Date: Oct. 16, 2014

(54) Compositions and Methods for Increasing Stress Resilience

(71) Applicant: The Children's Hospital of Philadelphia, Philadelphia, PA (US)

(72) Inventors: Seema Bhatnagar, Metuchen, NJ (US); Willem Heydendael, Philadelphia, PA (US); Abhishek Sengupta, Philadelphia, PA (US)

(21) Appl. No.: 14/357,381

(22) PCT Filed: Nov. 9, 2012
(86) PCT No.: PCT/US12/64387

§ 371 Date: May 9, 2014

Related U.S. Application Data
(60) Provisional application No. 61/558,674, filed on Nov. 11, 2011.

(51) Int. Cl. C07K 14/72; A61K 31/137; A61K 31/713

(52) U.S. Cl. CPC C07K 14/723; A61K 31/713; A61K 31/137
USPC 514/44 R; 514/653

(57) ABSTRACT

Compositions and methods for increasing resilience to the effects of stress are provided.

This application claims priority under 35 U.S.C. §119(e) to U.S. Provisional Patent Application No. 61/558,674, filed Nov. 11, 2011. The foregoing application is incorporated by reference herein.

This invention was made with government support under Grant No. ARO 58077 LS DRP awarded by the Defense Advanced Research Projects Agency. The Government has certain rights in this invention.

### FIELD OF THE INVENTION

The present invention relates to the field of stress resilience. Specifically, compositions and methods for inhibiting, treating, and/or preventing stress related disorders are disclosed.

### BACKGROUND OF THE INVENTION

Several publications and patent documents are cited throughout the specification in order to describe the state of the art to which this invention pertains. Each of these citations is incorporated herein by reference as though set forth in full.

Stress is associated with the onset of mood and anxiety disorders including post-traumatic stress disorder (PTSD) as well as exacerbation of symptoms of these disorders. Beyond psychiatric disorders, stress can impact the course of diseases in individuals, such as inflammatory bowel and other immunological diseases. Methods of increasing a subject's resistance or resilience to stress are desirable.

### SUMMARY OF THE INVENTION

In accordance with the present invention, compositions and methods for increasing stress resistance or resilience are provided. In accordance with an aspect of the instant invention, methods for inhibiting, treating, and/or preventing a stress-related disorder or symptoms thereof are provided. In a particular embodiment, the method comprises increasing sphingosine-1-phosphate receptor 3 (S1P3 receptor) expression and/or activity. In a particular embodiment, the method comprises administering to a subject at least one S1P3 receptor encoding nucleic acid or S1P3 agonist.

### BRIEF DESCRIPTIONS OF THE DRAWING

FIG. 1A provides a graph of sphingosine 1-phosphate receptor 3 mRNA (fold increase over control) from the prefrontal cortex of no defeat control rats (n=11), short defeat latency rats (SL; n=9), and long defeat latency rats (LL; n=10). FIG. 1B provides a graph of sphingosine 1-phosphate receptor 3 mRNA (fold increase) from the prefrontal cortex of rats as a function of the latency to be defeated.

FIG. 2A provides a graph of the defeat latency for short defeat latency mice (SL) or long defeat latency mice (LL) treated with vehicle or FTY720. *p=0.0726. FIG. 2B provides a graph of the social interaction time for control mice, SL mice, or LL mice treated with vehicle or FTY720. *p=0.0049.

### DETAILED DESCRIPTION OF THE INVENTION

A model of chronic social stress has been developed in rats in which subpopulations exhibit behavioral, neural and physiological indices of resilience or vulnerability to the effects of defeat (Wood et al. (2010) Endocrinology 151:1795-805). These subpopulations of rats exhibit varying behavioral phenotypes during defeat. In an otherwise homogeneous population of rats, a bimodal distribution became apparent in the average latency to become subordinate (defeated) over 7 days of defeat by an aggressive rat. Approximately half of the rats exhibited short defeat latencies (SL) whereas the other half of rats resisted defeat and exhibited increased defeat latency (long latency or LL). The sum of the behavioral, neuroendocrine and neural evidence led the finding that rats being defeated faster (SL rats) are more vulnerable and the rats actively resisting defeat (LL rats) are more resilient to the effects of stress. This is an important model as it allows for the identification of the neural substrates and factors of resilience and vulnerability and allows for the testing of potential therapeutic targets for stress-related diseases.

As demonstrated hereinbelow, it has been determined that there is increased expression of the gene for the immune system modulator sphingosine 1-phosphate receptor 3 in the prefrontal cortex of LL rats compared to SL rats and control rats. Further, there is a significant positive correlation between latency to be defeated and amount of expression of this gene. Thus, the longer the rats take to be defeated by an aggressive animal (i.e., the longer their latency), the higher the sphingosine 1-phosphate receptor 3 gene expression in the prefrontal cortex and the higher the resilience to stress.

Sphingolipids are a family of lipids that are essential in cell signaling, and in the structure of cell membranes and important in cell death and aspects of inflammation (Singh et al. (2008) J. Neurosci. Res., 86:1419-33). Sphingosine kinases catalyze phosphorylation of sphingosine to sphingosine-1-phosphate (Sph-1-P), a ubiquitous lipid mediator. Sph-1-P is an endogenous extracellular ligand for the sph-1-P (or EDG-1) family of 5 receptors G-protein coupled receptors. Sphingolipids are considered a possible approach to promote neuronal recovery from acute central nervous system (CNS) injury such as traumatic brain injury (TBI) and other neural traumas through actions on apoptosis and angiogenesis (Singh et al. (2008) J. Neurosci. Res., 86:1419-33). Here, the sphingosine-1-phosphate receptor 3 (S1P3 receptor) was increased in LL rats compared to control rats and SL rats.

The S1P3 receptor is a G protein-coupled receptor and is also known as endothelial differentiation gene 3 (EDG3). The nucleotide and amino acid sequences of the human S1P3 receptor can be found, for example, at GenBank Accession Nos. X83864, NM_{—}005226, and NP_{—}005217, and GeneID: 1903. S1P3 receptors couple to Gᵢ, G_{q}, and G_{12/13} (Rosen et al. (2009) Annu. Rev. Biochem., 78:743-768). The signal that converges from Gᵢ-coupled S1P receptors inhibits the activation of adenylate cyclase and induces the activation of p44/p42 mitogen-activated protein kinase (MAPK). S1P3 receptors mainly increase [Ca²⁺]ᵢ through the activation of phospholipase Cβ from G_{q} (Watterson et al. (2005) Cell Signal 17:289-298) and the S1P3 receptor plays an important role in the S1P-induced increase in [Ca²⁺]ᵢ (Ishii et al. (2002) J. Biol. Chem., 277:25152-25159). S1P3 receptor also couples to G_{12/13} protein to activate the small GTPase, Rho, which is involved in the regulation of actin-cytoskeleton (Ryu et al. (2002) Circ. Res., 90:325-332; Sugimoto et al. (2003) Mol. Cell. Biol., 23:1534-1545). S1P3 receptor is also central to S1P-regulated coronary flow (Murakami et al. (2010) Mol. Pharm., 77:704-713).

The instant invention encompasses methods of inhibiting, treating, and/or preventing stress related disorders and/or the symptoms associated therewith in a subject. In a particular embodiment, the subject is vulnerable to the effects of stress or has experienced an adverse reaction to stress previously (e.g., experienced a stress related event or trauma). The methods comprise administering increasing S1P3 expression and/or activity. In a particular embodiment, the stress related disorder is depression, post traumatic stress disorder (PTSD), or other forms of anxiety. The instant invention also encompasses methods of inhibiting, treating, and/or preventing disorders that are modulated (e.g., exacerbated) by stress, such as inflammatory bowel disorders, other immunological disorders, diabetes, hypertension, and cancer. The methods of the instant invention can be co-administered (sequentially and/or simultaneously) with at least one other therapeutic for the treatment of the stress related disorder (e.g., selective serotonin reuptake inhibitors (SSRIs) (e.g., sertraline (Zoloft®), paroxetine (Paxil®), or fluoxetine (Prozac®); noradrenergic drugs; monoamine oxidase inhibitors (MAOIs; e.g., ipronizad, phenylzine and pheniprazine); tricyclic antidepressants (TCAs; e.g., clomipramine, desipramine, imipramine, and serotonin); and norepinephrine re-uptake inhibitors (SSRIs; e.g., venalafazine, dluoxetine, and sibutramine).

In certain embodiments, the methods of the instant invention comprise increasing S1P3 receptor activity. In a particular embodiment, S1P3 receptor activity is increased by delivering/expressing nucleic acid molecules encoding S1P3 in cells. For example, nucleic acid molecules encoding S1P3 receptor (e.g., expression vectors (particularly viral vectors such as adenoviral vectors)) are delivered to the brain, particularly the prefrontal cortex. In a particular embodiment, the S1P3 encoding nucleic acid is under the control of a neuron specific promoter such as synapsin (e.g., Kugler et al. (2003) Gene Ther., 10:337-47).

In certain embodiments, the methods of the instant invention comprise administering at least one S1P3 receptor agonist and/or S1P3 nucleic acid to a subject. In a particular embodiment, the S1P3 receptor agonist and/or S1P3 nucleic acid is delivered as a composition with at least one pharmaceutically acceptable carrier. In certain embodiments, the S1P3 receptor agonist may also be an agonist of other S1P receptors. In certain embodiment, the S1P3 receptor agonist preferentially binds/activates the S1P3 receptor over other S1P receptors (e.g., a selective agonist). For example, the S1P3 receptor agonist may have an EC₅₀ that is at least 2-fold, at least 5-fold, at least 10-fold, at least 50-fold, at least 100-fold or more, lower with the S1P3 receptor than other S1P receptors. S1P3 receptor agonists include, without limitation, FTY720, sphingosine-1-phosphate, sphingosine analogues, 2-substituted 2-amino-propane-1,3-diol or 2-amino-propanol derivatives of sphingosine, (S)-phosphoric acid mono-[2-amino-3-(4-octyl-phenylamino)-propyl]ester (VPC 24191; Avanti® Polar Lipids, Inc., Alabaster, Ala.), (R)-phosphoric acid mono-[2-amino-2-(6-octyl-1H-benzoimiazol-2-yl)-ethyl]ester (VPC 23153, Avanti® Polar Lipids, Inc.), CID 2321431 (SID 3714904), CID 5309153 (SID 7967985), CID 665518 (SID 864271), CID 2842253 (SID 7977380), KM10340 (3-(6-tert-butyl-1,1-dimethyl-2,3-dihydro-1H-inden-4-yl)-5-(trifluoromethyl)-1H-pyrazole) and those agonists described in U.S. Patent Application Publication No. 2011/0124605; U.S. Pat. Nos. 7,842,685; 7,064,217; 7,208,502; 7,241,790; and 7,638,637; Shurer et al. (2008) ACS Chem. Biol., 3:486-498); Sammani et al. (2011) Am. J. Respir. Cell Mol. Biol., 45:1022-7; and “MLSCN Probe Summary: S1P3 Agonist” by The Scripps Research Institute Molecular Screening Center (available at mli.nih.gov/mli/?dl_id=721). In a particular embodiment, the S1P3 receptor agonist is FTY720.

FTY720 (also known as fingolimod) is a modulator of Sph-1-P (Foster et al. (2007) J. Pharmacol. Exp. Ther., 323:469-75). FTY720 is an active metabolite of the sphingosine pathway and acts as a potent agonist at multiple Sph receptors, including the S1P3 receptor. Indeed, FTY720 acts as a prodrug which is phosphorylated in vivo and the phosphorylated derivative is an agonist for S1P1, S1P3, S1P4, and S1P5 receptors, but not the S1P2 receptor (Chiba et al. (2005) Pharmacol. Therap., 108:308-319).

FTY720 has recently been approved for treating multiple sclerosis. Further, amyloid protein is able to induce proinflammatory effects in part by attracting monocytes and cytokines, and this process is sphingosine-kinase dependent and involves sphingosine receptors (Mielke et al. (2010) Neuromolecular Med., 12:331-40; Kaneider et al. (2004) FASEB J., 18:1309-11). FTY720 arrests their migration towards amyloid and, as such, is a target for delaying plaque formation in Alzheimer's (Kaneider et al. (2004) FASEB J., 18:1309-11).

S1P3 receptor agonists may also be identified by assays known in the art, such as through high throughput screening. For example, compounds (e.g., small molecules) may be assayed in the S1P3 Agonist Primary HTS and Confirmation Assay by The Scripps Research Institute Molecular Screening Center (see PubChem BioAssay identifier (AID)=373; pubchem.ncbi.nlm. nih.gov/assay/assay.cgi?aid=373).

As stated hereinabove, the instant invention encompasses methods of inhibiting, treating, and/or preventing stress related disorders and/or the symptoms associated therewith in a subject. The method may further comprise diagnosing a stress related disorder in the subject prior to administration (see, e.g., DSM-IV-TR). Stress may be considered a psychological and/or physical condition which comes as a result of physical or/and mental “pressure”, which overwhelms adaptive capacities. The methods of the instant invention mediate resilience to the effects of stress. The S1P3 receptor agonists may be administered to a subject in order to treat or inhibit (e.g., reduce) stress in a subject or prevent the onset of stress. In certain embodiments, the S1P3 receptor agonists are administered to a subject who has a predisposition to stress. In certain embodiments, the S1P3 receptor agonists are administered to a subject prior to, after, or at the same time as exposure to a stress-inducing event (e.g., exposure to a stressor such as trauma or trauma reminders). For example, the S1P3 receptor agonist may be administered within 1 day of the stress-inducing event or within 12 hours, 6 hours, 3 hours, 2 hours, 1 hour, or less of the stress-inducing event. In a particular embodiment, administration occurs prior to the stress-inducing event.

The methods of the instant invention encompass inhibiting, treating, and/or preventing the symptoms associated with stress related disorders. For example, the administration of S1P3 receptor agonists may treat, inhibit, and/or prevent at least one symptom associated with a stress related disorder such as irregular sleep architecture, irregular circadian rhythms in core temperature, failures in behavioral tests of anxiety, and depressive-like behavior.

As used herein, the term “symptom” may generally refer to subjective indications that characterize a disorder. For example, symptoms of post-traumatic stress disorder include, without limitation: recurrent and intrusive trauma recollections, recurrent and distressing dreams of the traumatic event, acting or feeling as if the traumatic event were recurring, distress when exposed to trauma reminders, physiological reactivity when exposed to trauma reminders, efforts to avoid thoughts or feelings associated with the trauma, efforts to avoid activities or situations, inability to recall trauma or trauma aspects, markedly diminished interest in significant activities, feelings of detachment or estrangement from others, restricted range of affect, sense of a foreshortened future, social anxiety, anxiety (particularly with unfamiliar surroundings), difficulty falling or staying asleep, irritability or outbursts of anger, difficulty concentrating, hypervigilance, exaggerated startle response, abnormal respiration, abnormal cardiac rate of rhythm, abnormal blood pressure, abnormal function of a special sense, and abnormal function of sensory organ. Certain of these symptoms may also be symptoms of other stress related disorders.

“Stress related disorders” may refer collectively to maladies characterized by a state of hyper- or hypo-arousal with hyper- and hypo-vigilance. Stress related disorders include, without limitation: depression, major depressive disorder (MDD), anxiety disorder, panic disorder (episodic paroxysmal anxiety), panic attack, obsessive compulsive disorder, social anxiety disorder, phobic anxiety disorders (e.g., acrophobia, claustrophobia, agoraphobia, social phobia, and other phobias), posttraumatic stress disorder (PTSD), acute stress disorder, and obsessive compulsive disorder. Stress-related disorders may also include non-psychiatric disorders such as hypertension, inflammatory bowel disorders, other immunological disorders, diabetes, and cancer.

The compositions of the present invention can be administered by any suitable route, for example, by injection (e.g., for local, direct, or systemic administration), oral, pulmonary, topical, nasal or other modes of administration. The composition may be administered by any suitable means, including parenteral, intramuscular, intravenous, intraarterial, intraperitoneal, subcutaneous, topical, inhalatory, transdermal, intrapulmonary, intraareterial, intrarectal, intramuscular, and intranasal administration. In a particular embodiment, the composition is administered intraperitoneally. In a particular embodiment, the composition is administered directly to neurons and/or the brain (e.g., non-systemic delivery). In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., Tween 80, Polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention (see, e.g., Remington's Pharmaceutical Sciences and Remington: The Science and Practice of Pharmacy). The pharmaceutical composition of the present invention can be prepared, for example, in liquid form, or can be in dried powder form (e.g., lyophilized for later reconstitution).

The therapeutic agents described herein (e.g., S1P3 receptor nucleic acid molecules or agonists) will generally be administered to a patient as a pharmaceutical preparation. The term “patient” as used herein refers to human or animal subjects. The compositions of the instant invention may be employed therapeutically or prophylactically, under the guidance of a physician.

The compositions comprising the agent of the instant invention may be conveniently formulated for administration with any pharmaceutically acceptable carrier(s). The concentration of agent in the chosen medium may be varied and the medium may be chosen based on the desired route of administration of the pharmaceutical preparation. Except insofar as any conventional media or agent is incompatible with the agent to be administered, its use in the pharmaceutical preparation is contemplated.

The dose and dosage regimen of the agent according to the invention that is suitable for administration to a particular patient may be determined by a physician considering the patient's age, sex, weight, general medical condition, and the specific condition for which the agent is being administered to be treated or prevented and the severity thereof. The physician may also take into account the route of administration, the pharmaceutical carrier, and the agent's biological activity. Selection of a suitable pharmaceutical preparation will also depend upon the mode of administration chosen.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment or prevention therapy. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation or prevention of a particular condition may be determined by dosage concentration curve calculations, as known in the art.

The pharmaceutical preparation comprising the agent may be administered at appropriate intervals, for example, at least twice a day or more until the pathological symptoms are reduced or alleviated, after which the dosage may be reduced to a maintenance level. The appropriate interval in a particular case would normally depend on the condition of the patient. With regard to prevention or reduction of stress, the compositions of the instant invention may be administered in doses at appropriate intervals prior to exposure to the stress stimuli.

Toxicity and efficacy (e.g., therapeutic, preventative) of the particular formulas described herein can be determined by standard pharmaceutical procedures such as, without limitation, in vitro, in cell cultures, ex vivo, or on experimental animals. The data obtained from these studies can be used in formulating a range of dosage for use in human. The dosage may vary depending upon form and route of administration. Dosage amount and interval may be adjusted individually to levels of the active ingredient which are sufficient to deliver a therapeutically or prophylactically effective amount.

### Definitions

The following definitions are provided to facilitate an understanding of the present invention:

The singular forms “a,” “an,” and “the” include plural referents unless the context clearly dictates otherwise.

As used herein, the term “agonist” refers to an agent (e.g., protein, polypeptide, peptide, lipid, antibody, antibody fragment, large molecule, or small molecule) that binds to a receptor and has an intrinsic effect such as inducing a receptor-mediated response. For example, the agonist may stimulate, increase, activate, facilitate, enhance, or up regulate the activity of the receptor.

The term “post-traumatic stress disorder” refers to a psychiatric condition in its broadest sense characterized by persistent re-experiencing of an extreme traumatic event. “Post-traumatic stress disorder” may be defined as in the American Psychiatric Association: Diagnostic and Statistical Manual of Mental Disorders, Fourth Edition, Text Revision, Washington, D.C., 2000 (DSM-IV-TR).

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions may be employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., stress related disorder) resulting in a decrease in the probability that the subject will develop the condition.

A “therapeutically effective amount” of a compound or a pharmaceutical composition refers to an amount effective to prevent, inhibit, or treat a particular disorder or disease and/or the symptoms thereof. For example, “therapeutically effective amount” may refer to an amount sufficient to modulate stress and/or stress response in a subject.

As used herein, the term “subject” refers to an animal, particularly a mammal, particularly a human.

As used herein, “stressor” refers to any stimulus that causes a stress reaction in a living subject. The stressor may be an external stimulus. Examples of stressors include, without limitation, sensory inputs (e.g., pain, bright light, noise, and the like), trauma, conflict, social, interpersonal, cognitive, and the like.

The following examples provide illustrative methods of practicing the instant invention and are not intended to limit the scope of the invention in any way.

### EXAMPLE 1

### Materials and Methods

### Animals

Male Sprague Dawley rats (275-300 g on experiment day 1) were used as controls or intruders, and male Long-Evans retired breeders (650-850 g) served as residents (Charles River, Wilmington, Mass.). Rats were singly housed with a 12-hour light, 12-hour dark cycle (lights on at 0700 h) in a climate-controlled room with ad libitum food and water. Studies were approved by the Children's Hospital of Philadelphia Institutional Animal Care and Use Committee and conformed to the National Institutes of Health Guide for the Use of Laboratory Animals. All experimentation was conducted between 0900 and 1200 h.

### Social Defeat

The social defeat paradigm used in these studies was modified from the resident-intruder model (Miczek, K. A. (1979) Psychopharmacology (Berl) 60:253-259). Rats were randomly assigned to either a social defeat or control group for a consecutive 7 days (Bhatnagar et al. (2006) J. Neuroendocrinol., 18:13-24; Buwalda et al. (1999) J. Neuroendocrinol., 11:513-520). During each episode of social stress, a rat was placed into the home cage territory of an unfamiliar Long-Evans resident previously screened for high aggression (Bhatnagar et al. (2006) J. Neuroendocrinol., 18:13-24; Buwalda et al. (1999) J. Neuroendocrinol., 11:513-520). A typical agonistic encounter resulted in intruder subordination or defeat, signaled by the intruder assuming a supine position for approximately 3 seconds. After defeat, a wire mesh enclosure was placed in the cage to prevent physical contact between the resident and intruder but allowing visual, auditory, and olfactory contact for the remainder of the 30-minute defeat session. Latency to assume a submissive posture (defeat) was recorded and averaged over the seven daily defeat exposures. If an intruder resisted defeat for 15 minutes, rats were separated with the wire partition for the remainder of the session. Controls were placed behind a wire partition in a novel cage for 30 minutes daily. Rats were returned to their home cage after each session, and body weight was recorded on days 1 and 8.

### Experimental Design

All rats were assigned into either a control or defeat stress group. The stress protocol was kept constant in all experiments such that rats were exposed to a consecutive 7 days of a 30-minute social defeat or control novel cage exposure.

### Sphingosine 1-Phosphate Receptor 3 Gene Expression

Three groups of rats were used: no defeat controls (n=11), rats showing short latency to be defeated over the course of 7 days (SL rats; n=9) and rats showing long latency to be defeated over the course of 7 days (LL rats; n=10). mRNA levels of sphingosine 1-phosphate receptor 3 were measured in the prefrontal cortex of these animals.

### Results

As explained in Wood et al. (Neuroendocrinol. (2010) 151:1795-1805), a model of chromic social stress has been developed. Using this model, a bimodal distribution emerged in an otherwise homogeneous population of Sprague Dawley rats such that 42% of rats exhibited short defeat latencies (SL rats; <300 seconds), whereas 58% of rats resisted defeat and exhibited longer latencies (LL rats; >300 seconds). These two phenotypes were associated with distinct endocrine profiles, distinct behavioral profiles, and differences in components of the corticotropin-releasing factor (CRF) system. The short-latency subpopulation exhibited hypothalamic-pituitary-adrenal (HPA) dysregulation and behavior similar to that observed in melancholic depression. Examination of components of the CRF system indicate that proactive behavior in resisting defeat exhibited by long-latency rats was associated with decreased efficacy of CRF. Together, these data indicate that inherent differences in stress reactivity, perhaps as a result of differences in CRF regulation, can predict long-term consequences of social stress and vulnerability to depressive-like symptoms. Using the above model of social defeat, genes within the prefrontal cortex that differed between SL and LL rats were identified. The prefrontal cortex was focused on because this structure mediates executive functions in humans and is key for emotional regulation. Changes in expression were observed between SL and LL rat genes. In particular, changes were observed with the sphingosine 1-phosphate receptor 3 gene (Sph-1-P-3).

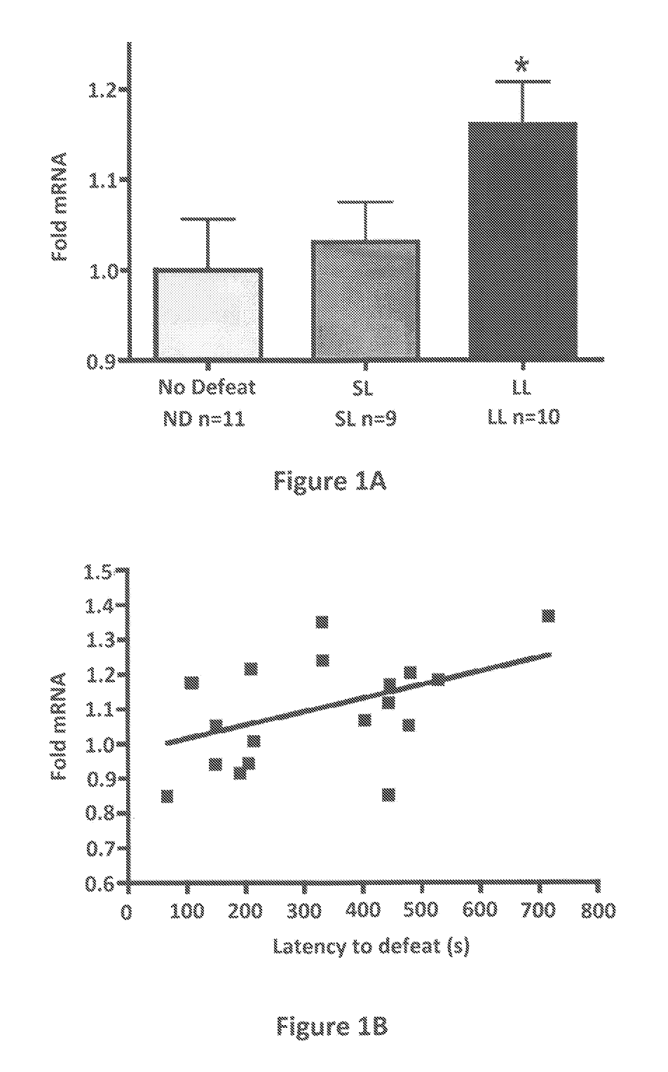

FIG. 1 shows that the Sph-1-P-3 gene is expressed at higher levels in the resilient LL rats compared to vulnerable SL rats or control rats. This indicates that increased expression of this gene is associated with stress resilience. Furthermore, the expression is significantly correlated with the latency to be defeated showing that it increases the more an animal is resistant to social defeat (as their latency to be defeated increases). Identification of this gene indicates that resilience to the effects of chronic stress is related to increased expression of this gene and that this gene represents a novel target for increasing stress resilience. Indeed, drugs targeting this receptor will modulate stress effects on psychiatric and other diseases. More particularly, agonists to this receptor will promote stress resilience.

### EXAMPLE 2

Animals were exposed to 7 days of social defeat stress. During these 7 days of defeat stress the average latency to defeat was observed. Based on this latency to defeat, animals were either classified as short latency (SL, defeat <300 seconds) or long latency (LL, defeat >300 seconds). SL animals exhibit passive coping behaviors and are vulnerable to the long-term effects of the social defeat stress, such as increases in depressive type behaviors and decreased social interaction time in the social interaction test which is also indicative of an increase in anxiety. LL animals, however, exhibit active coping strategies and exhibit resilience to the long-term effects of the social defeat stress.

Following 7 days of defeat, the SL and LL groups were divided into two separate treatment groups receiving either vehicle or FTY720, 1 hour prior to social defeat stress on the following 3 days. Defeat latencies were recorded. Additionally, 48 hours following the end of social defeat stress, animals were tested in the social interaction test of anxiety.

FTY720, also known as fingolimod and Gilenya™ (Novartis), is a sphingosine phosphate analogue. It is thought to decrease inflammation by sequestering lymphocytes in the lymph nodes. However, the sphingosine phosphate family of receptors is widely distributed throughout the central nervous system (CNS) and may present other potential modes of action for the FTY720 compound.

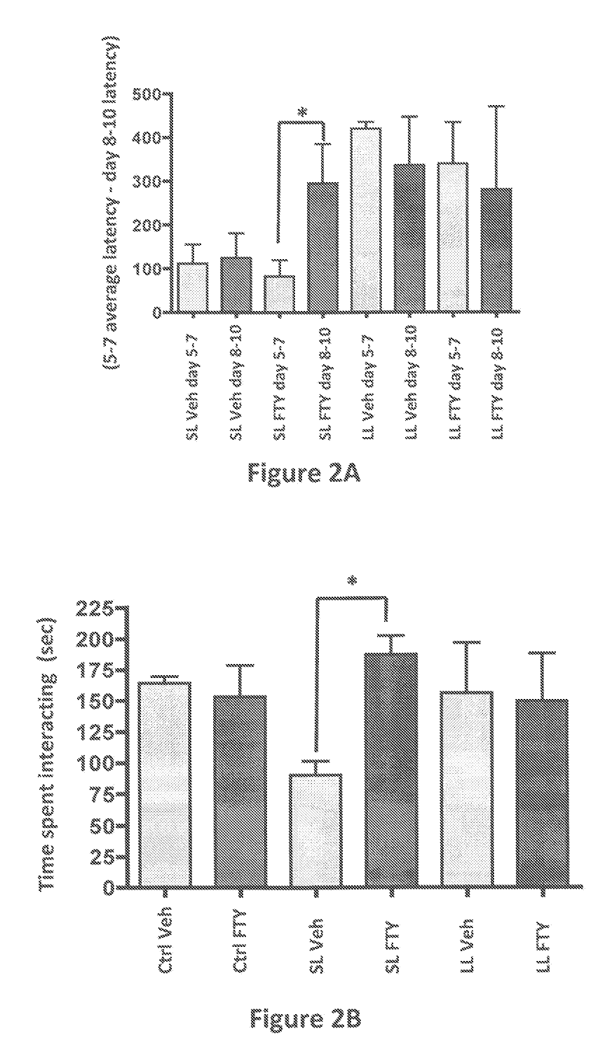

When screened for activity in the social defeat paradigm, FTY720 tended to increase defeat latency of SL animals to the level of LL animals (FIG. 2A; *p=0.0726). This finding indicates that the behavior exhibited by vulnerable rats during the stress experience itself can be reversed towards a more resilient behavioral phenotype. In the social interaction test, FTY720 prevented the decreased interaction time typically exhibited by SL animals (FIG. 2B, *p=0.0049). This finding indicates that FTY720 can prevent the increased anxiety exhibited by vulnerable rats resulting in a reduced anxiety level similar to the LL resilient rats. The drug had no effects in the resilient LL rats or in control rats. These results indicate that not only can FTY720 alter the coping strategy during the stress experience itself, from passive to active, but also prevent the long-term effects of the social defeat stress on anxiety, changing a vulnerable phenotype into a resilient phenotype.

In sum, these results show that a drug targeted to sphingosine receptors can modify resilience to stress. More specifically, a drug that stimulates sphingosine receptors produces resilience to the anxiety-inducing effects of stress. While FTY720 is a non-specific analog of sphingosine phosphate and acts at multiple receptors for sphingosine phosphates, these results clearly show that sphingosine receptors are important for producing resilience to stress.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for preventing or treating a stress-related disorder or symptoms thereof in a subject, said method comprising administering to said subject a nucleic acid molecule encoding sphingosine-1-phosphate receptor 3 (S1P3 receptor) and/or at least one S1P3 receptor agonist.

2. The method of claim 1, wherein said method comprises administering to said subject at least one S1P3 receptor agonist.

3. The method of claim 2, wherein said S1P3 receptor agonist is FTY720.

4. The method of claim 1, wherein said stress-related disorder is selected from the group consisting of depression, post-traumatic stress disorder, and anxiety disorders.

5. The method of claim 4, wherein said stress-related disorder is post-traumatic stress disorder.

6. The method of claim 1, wherein said method comprises preventing or treating the symptoms associated with said stress-related disorder.

7. The method of claim 1, wherein said S1P3 receptor agonist is administered to the subject prior to a stress inducing event.

8. The method of claim 7, wherein said S1P3 receptor agonist is administered within 1 hour of the stress inducing event.

9. A method for inhibiting stress in a subject, said method comprising administering to said subject a nucleic acid molecule encoding sphingosine-1-phosphate receptor 3 (S1P3 receptor) and/or at least one S1P3 receptor agonist.

10. The method of claim 9, wherein said method comprises administering to said subject at least one S1P3 receptor agonist.

11. The method of claim 10, wherein said S1P3 receptor agonist is FTY720.

12. The method of claim 9, wherein said subject has a stress-related disorder.

13. The method of claim 12, wherein said stress-related disorder is selected from the group consisting of depression, post-traumatic stress disorder, and anxiety disorders.

14. The method of claim 9, wherein said S1P3 receptor agonist is administered to the subject prior to a stress inducing event.

15. The method of claim 14, wherein said S1P3 receptor agonist is administered within 1 hour of the stress inducing event.
